(19) United States
(12) Patent Application Publication
(10) Pub. No.: US 2013/0123636 A1
(43) Pub. Date: May 16, 2013

(54) Methods and Devices For Proper Stabilization Of Fetal Monitoring Devices

(71) Applicant: Melanie Longmore, Draper, UT (US)

(72) Inventor: Melanie Longmore, Draper, UT (US)

(73) Assignee: INTERMOUNTAIN INVENTION MANAGEMENT, LLC, Salt Lake City, UT (US)

(21) Appl. No.: 13/810,636

(22) PCT Filed: Jul. 18, 2011
(86) PCT No.: PCT/US11/01275

§ 371 Date: Jan. 16, 2013

Related U.S. Application Data
(60) Provisional application No. 61/399,775, filed on Jul. 16, 2010.

(51) Int. Cl. A61B 8/00; A61B 8/08; A61B 8/02

(52) U.S. Cl. CPC A61B 8/4227; A61B 8/02; A61B 8/4477; A61B 8/0866
USPC 600/453; 600/459

(57) ABSTRACT

Certain embodiments of the present invention provide fetal monitoring belt devices. The fetal monitoring belt devices include belts and a back support. In certain embodiments, the belts may further include one or more rows of buttonholes to provide multiple places for attachment of one or more ultrasound transducers via buttonhooks. In certain embodiments, the back support may further include an interior pouch to hold a therapeutic device, such as a hot or cold pack and/or a vibration element. The fetal monitoring belt devices of the present invention provide a comfortable, long-term, stable way of maintaining the proper positioning of ultrasound transducers during prenatal exams, labor and delivery.

### FIELD OF THE INVENTION

The present invention relates generally to fetal monitoring belt devices. More specifically, the present invention pertains to improved fetal monitoring belt devices including a back support to comfortably position and stabilize ultrasound transducers on women during prenatal check-ups, labor and delivery.

### BACKGROUND

Practitioners and hospitals routinely use external fetal monitoring during labor or delivery as a way of measuring both uterine contractions and fetal heart rate. It is also routinely used during prenatal check-ups throughout a woman's pregnancy. It is an ideal way of non-invasively monitoring both the mother and fetus prior to labor and then throughout labor and delivery.

Ultrasound machines are often used to electronically monitor and process signals transmitted from transducers positioned on the patient. For example, during labor, the ultrasound system continuously monitors and records the fetal heart rate and uterine contractions. The transducers typically connect either by wireless telemetry or by a hard-wired cable directly to the recording and monitoring machine.

The accuracy of the reading produced by any monitoring technique relies on the proper placement of the external sensors or transducers. Typically, there will be one or more transducers to monitor each fetal heart rate (ultrasound transducer) and a separate one to monitor uterine contractions (tocodynamometer, or toco transducer). Each of the transducer's position is held in place on the patient's abdomen by a belt that circles the torso and the belts typically include one or more pieces of flexible material adapted to accommodate the curves of the maternal midsection.

The most common method of hooking the transducers to the belts is by hooking the button-hooks attached to each transducer directly to the belts via interspaced holes (“buttonholes”) along the length of the belts. Using this method, both ends of the belt can be fixably attached to the transducer simply by hooking each buttonhook through a buttonhole on each belt. Another method utilizes belts with hook and loop fasteners (commonly referred to as Velcro) which can be fastened to the transducers via solid metal belt loops designed into the sides of the transducers. For this method, each end of the belt has a hook and loop closure which can be tightened to optimally fit the patient.

In order to produce readings during labor, the health care professional must constantly reposition the transducers or hold them in place steadily as both the patient and fetus change position during labor causing the transducers to shift position and produce inaccurate readings. Additionally, existing belts tend to twist or otherwise change position, which also causes the transducers to change position or flip producing inaccurate readings and produces extreme discomfort for the patient.

The problem of properly positioning the transducers to get an accurate reading is greatly exacerbated in particular patient populations, such as in women who are overweight, delivering preterm and/or those with multiple gestations which require multiple ultrasound transducers. Therefore, there is a need for an improved fetal monitoring belt device that is comfortable for the patient and also allows a medical professional to maintain the appropriate angle of one or more transducers for a prolonged period of time, such as during prenatal check-ups, labor and delivery.

### SUMMARY OF THE INVENTION

It is therefore an object of the present invention to provide a new and improved fetal monitoring belt device, which overcomes all of the aforementioned disadvantages.

It is another object of the present invention to provide a new and improved fetal monitoring belt device which includes a back support and belts used to properly position ultrasound transducers on a patient during prenatal exams, labor and delivery.

In certain embodiments, the back support is made from a material selected from cotton, canvas, leather, bandaging, foam, solid formed gel, injection-molded plastic or biodegradable materials.

In a certain embodiment, the belts have a width and a thickness sufficient to maintain its cross-sectional shape while under tension and wrapped around a patient. In a particular embodiment, the belts range from three to twenty inches in width. In another particular embodiment, the belts range from sixteen to seventy-five inches in length. In an additional embodiment, the belts are labeled with an item selected from the group consisting of instructions, advertisements, sizing and color. In another particular embodiment, the belts are anchored to or threaded through said back support. In yet another particular embodiment, the belts are threaded through loops on the sides of transducers and have hook and loop materials on the ends allowing for optimal placement of transducers.

Another object of certain embodiments of the present invention is to provide belts with buttonholes spaced along the length of the belt.

Yet another object of the present invention is to provide belts have multiple rows of buttonholes.

Other objects of certain embodiments of the present invention relate to a fetal monitoring device having a therapeutic device which may include a hot or cold pack or vibration element.

Yet another object of the present invention is to provide transducers with buttonhooks allowing for direct skin contact with a patient and precise positioning for monitoring uterine contractions or a fetal heartbeat by threading said buttonhooks through said buttonholes in said belts. In a particular embodiment, the belts of the present invention have more than three transducers positioned on them to allow for the monitoring of multiple fetal heart rates.

These together with other objects and advantages of the present invention, along with the various features of novelty which characterize the invention, are pointed out with particularity in the claims annexed to and forming a part of this disclosure. The invention is capable of other embodiments and of being practiced or being carried out in various ways. Further objectives of the present invention will become apparent from a consideration of the drawings and ensuing description. As such, for a better understanding of the present invention, its operating advantages and the specific objects attained by its uses, reference should be made to the accompanying drawings and detailed description illustrating particular embodiments of the present invention.

### BRIEF DESCRIPTION OF THE DRAWINGS

With specific reference now to the figures in detail, it is stressed that the particulars illustrated in each figure are shown by way of example only and are merely for purposes of illustrative discussion of particular embodiments of the present invention. They are merely presented to provide useful and readily understood description of the principles and conceptual aspects of the invention. In this regard, no attempt is made to show structural details of the invention in more detail than is necessary to gain a fundamental understanding of the invention, the description taken together with the drawings will make apparent to those skilled in the art how the invention works in practice.

FIG. 1 is a perspective view of the fetal monitoring belt device according to certain embodiments of the present invention.

FIG. 2 is a perspective view of the fetal monitoring belt device including multiple rows of buttonholes for attachment of one or more fetal monitoring devices according to certain embodiments of the present invention.

FIG. 3 is a perspective view of the fetal monitoring belt device including an interior pouch for a therapeutic device, such as a hot or cold pack or vibration element, according to certain embodiments of the present invention.

FIG. 4 is a close-up view of an exemplary belt with multiple rows of buttonholes according to certain embodiments of the present invention.

FIG. 5 illustrates a fetal monitoring belt device including exemplary placement of transducers on a patient that are connected by a first and second lead to a monitor according to certain embodiments of the present invention.

FIG. 6 illustrates a front view of a fetal monitoring belt device on a patient including exemplary placement of transducers that are connected by a first and second lead to a monitor (not shown) according to certain embodiments of the present invention.

FIG. 7 illustrates a front view of a fetal monitoring belt device on a patient carrying twins including exemplary placement of two transducers, each monitoring an individual fetal heart rate and one transducer monitoring uterine contractions according to certain embodiments of the present invention. One transducer monitoring a fetal heart rate and the transducer monitoring uterine contractions are connected by a first and second lead to one monitor (not shown). The second transducer monitoring the second fetal heart rate is connected by a third lead to a second monitor (not shown).

FIG. 8 illustrates a perspective view of the fetal monitoring belt device illustrating the belts in a crossed position with one belt in front of the other according to an embodiment of the present invention.

FIG. 9 illustrates another perspective view of the fetal monitoring belt device illustrating the belts in a crossed position with one belt in front of the other according to an embodiment of the present invention.

FIG. 10 illustrates another perspective view of the fetal monitoring belt device illustrating the belts in an alternative crossed position with one belt in front of the other according to an embodiment of the present invention.

### DETAILED DESCRIPTION OF THE INVENTION

The devices of the present invention provide a comfortable, precise and stable solution to the problem of having to maintain continuous skin contact and the appropriate angle for transducers during prenatal exams, labor or delivery. The belts of the present invention are wide enough to distribute the tension evenly around a patient to prevent ultrasound transducers from losing contact with a patient's skin or otherwise changing position. Additionally, the back support of the present invention further provides stability and support to the belts of the present invention to further assist in distributing tension evenly along the length of each belt to prevent twisting and transducer displacement. The devices of the present invention are also comfortable to a patient undergoing monitoring for prenatal exams, labor or delivery.

In particular patient populations, such as in women who are overweight or obese, or those with multiple gestations, proper positioning of one or more transducers is particularly difficult due to the large abdomen and/or excess folds of adipose tissue associated with these conditions that can create an uneven surface for the transducer and one or more belts. The large size of the abdomen and/or excess adipose tissue often causes the transducer(s) and belt(s) to twist and/or otherwise change position excessively in light of the steep grade of the extended belly associated with multiple gestations or fat folds associated with an overweight or obese mother. Devices of the present invention may be used to create a stable surface for the transducer in any patient, including those with multiple gestations or excess fat and also provide increased patient comfort.

The devices of the present invention further provide a relatively stable and continuous reading from a transducer in all patients as the appropriate skin contact is maintained, as well as the appropriate angle needed to produce a consistent reading. This allows medical professionals to continually monitor both the patient and the fetus with fewer or no interruptions in the recording due to the transducer becoming displaced.

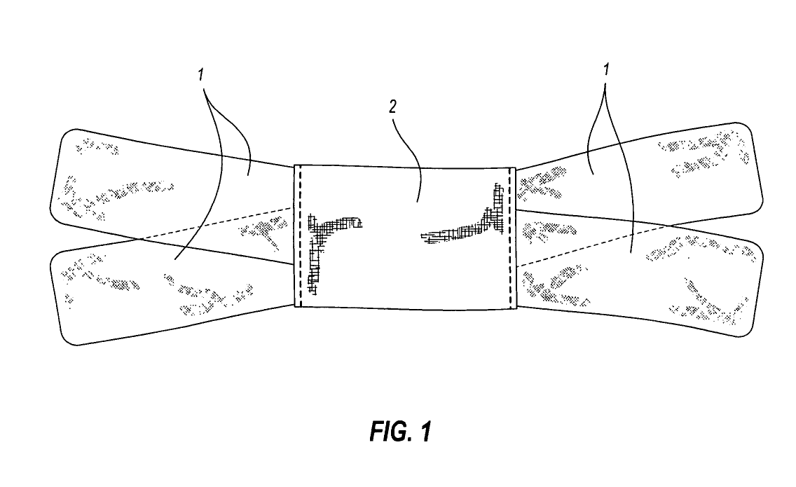

FIGS. 1 through 3 illustrate certain embodiments of the present invention that include one or more belts 1 and a back support 2. The belts and back support encircle a patient. The belts 1 may be anchored to the back support 2 or threaded through it. The length and width of the belt will be determined by what is necessary to provide sufficient tension along the length of the belt to prevent the belt from twisting causing patient discomfort and transducer displacement. The length of the belts will also vary depending on whether they are threaded through the back support or anchored to it prior to the fetal monitoring device encircling a patient. If they are anchored to the back support itself, they may be shorter than if they are threaded through the back support. Those skilled in the art will understand how to determine the appropriate width and length of the belts of the present invention depending on whether the belts are anchored to the back support or threaded through it in coordination with the needs of a particular patient. In one embodiment of the present invention, each individual belt may be in the range of sixteen (16) inches to seventy-five (75) inches in length. In a particular embodiment, the belts are twenty-five (25) inches in length. In another embodiment of the present invention, the belts 1 may be in the range of three (3) inches to twenty (20) inches in width. In a particular embodiment, the belts 1 are four (4) inches in width.

The belts 1 may be made of any material. In a certain embodiment, the belts 1 are made of a material capable of reuse after sterilization. In another embodiment, they are made of a material that is disposable and designed to be thrown away after a single use. The belts 1 of the present invention may also be made of a material that is either waterproof or insoluble in a variety of solutions, such as water, ultrasound gel, blood, alcohol and the like.

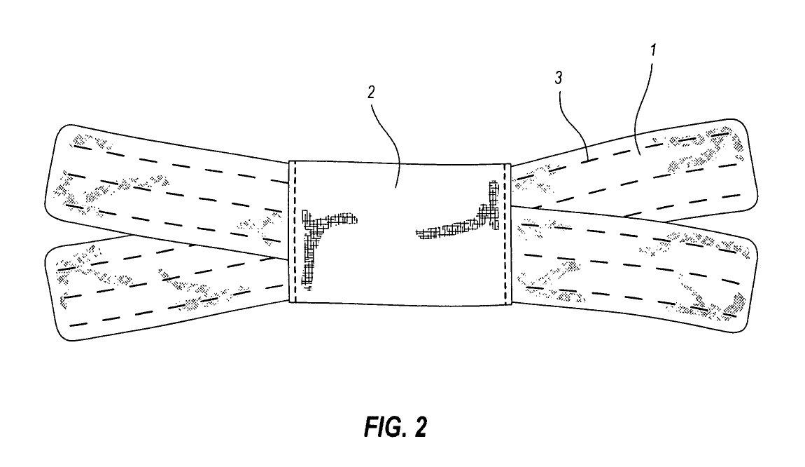

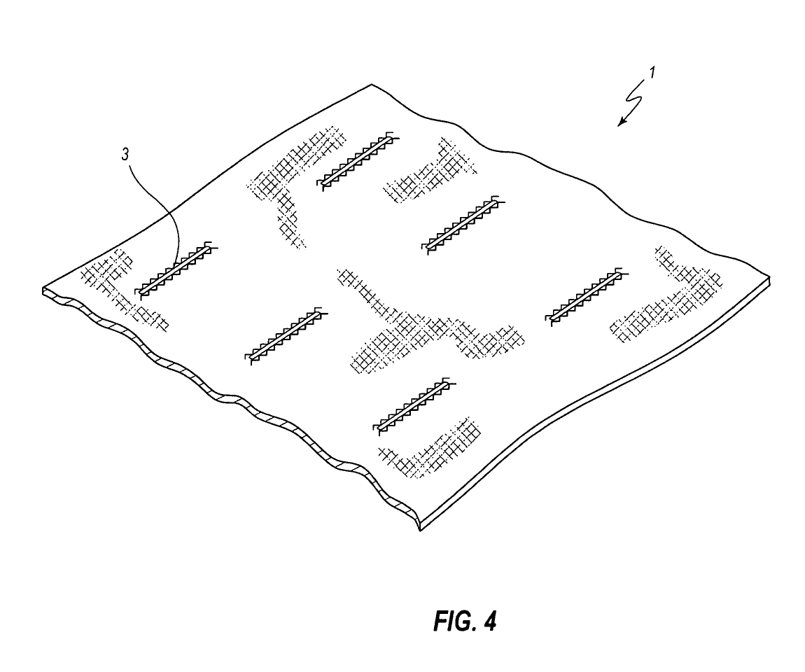

The belts 1 may include multiple rows of buttonholes 3 along the length of the belt for attachment of one or more transducers 4. Although FIGS. 2 through 4 illustrate three rows of buttonholes 3, the belts 1 may include any number of rows of buttonholes including one row or more than three rows of buttonholes. The buttonholes 3 of the present invention may be of any shape and size deemed appropriate for the button-hooks 5 of the transducers 4 used in the present invention. The buttonholes 3 of the present invention may have no reinforcement whatsoever and may merely be apertures through the belts themselves. They may also be reinforced with stitching as illustrated in FIG. 4 or may have strips of reinforcement material secured to the holes.

The belts 1 of the present invention can be rapidly adjusted, removed and/or reapplied as necessary. Their placement and tension can also be continually adjusted to maintain the appropriate angle for one or more transducers and to provide comfort to the patient.

The belts 1 of the present invention may be printed with end-user instructions, advertisements, sizing, and/or any other information helpful to the healthcare provider assisting in the monitoring of the patient's health. The belts 1 of the present invention may also be color-coded to distinguish between different lengths and/or to distinguish between belts having different size or types of buttonholes 3. The belts 1 of the present invention may also be color-coded to distinguish the sex of the fetus.

The transducers 4 of the present invention may be related to any ultrasound machine 7 capable of monitoring the health of a mother or fetus. By no means an exhaustive list, the following list represents an exemplary list of transducers understood in the art and such transducers include, but are not limited to, the hard-wired type as described by Quedens et al. in U.S. Pat. No. 5,373,843 issued on Dec. 20, 1994 or the type described by Combs in U.S. Pat. No. 6,151,520 issued on Nov. 21, 2000. Other types of transducers include such devices as described by Hon et al. in U.S. Pat. No. 4,920,966 issued on May 1, 1990. All these aforementioned references are included by reference as if fully set forth herein. Accordingly, it will be appreciated by those skilled in the art that various configurations of the present invention may be tailored to better suit a particular transducer. Any ultrasound machine may be used with the present invention. Examples of fetal monitoring machines include, but are not limited to, the system described in U.S. Pat. No. 4,781,200 to Baker issued on Nov. 1, 1988. Another example of a fetal monitoring machine is brand Corometrics 250-series model number 0172WAT-B manufactured by GE Healthcare Technologies of Waukesha, Wis. USA. Other machines made by Hewlett-Packard, for example, are also contemplated.

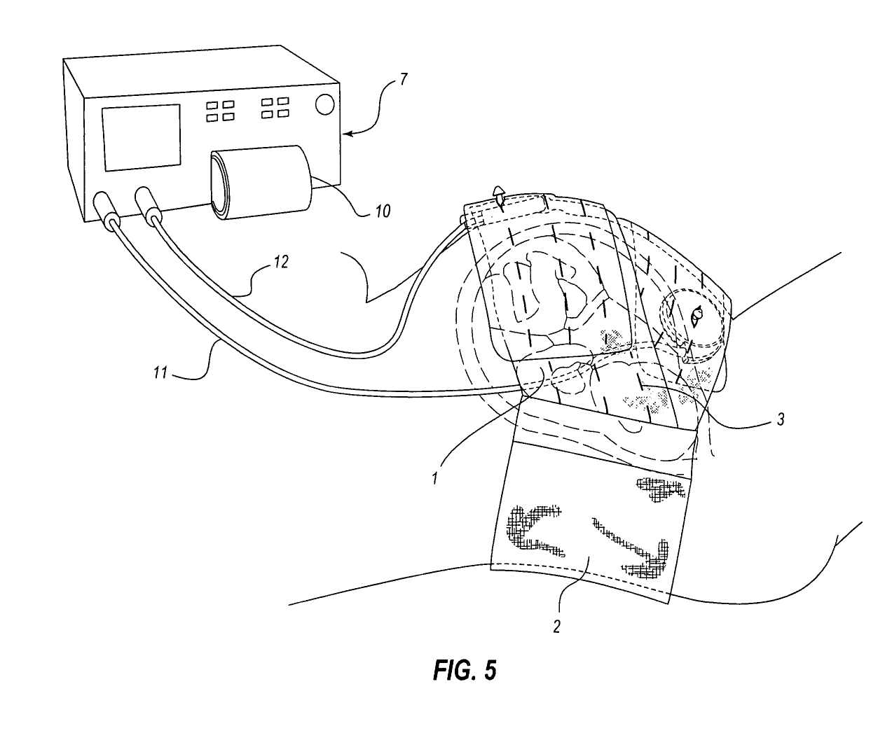

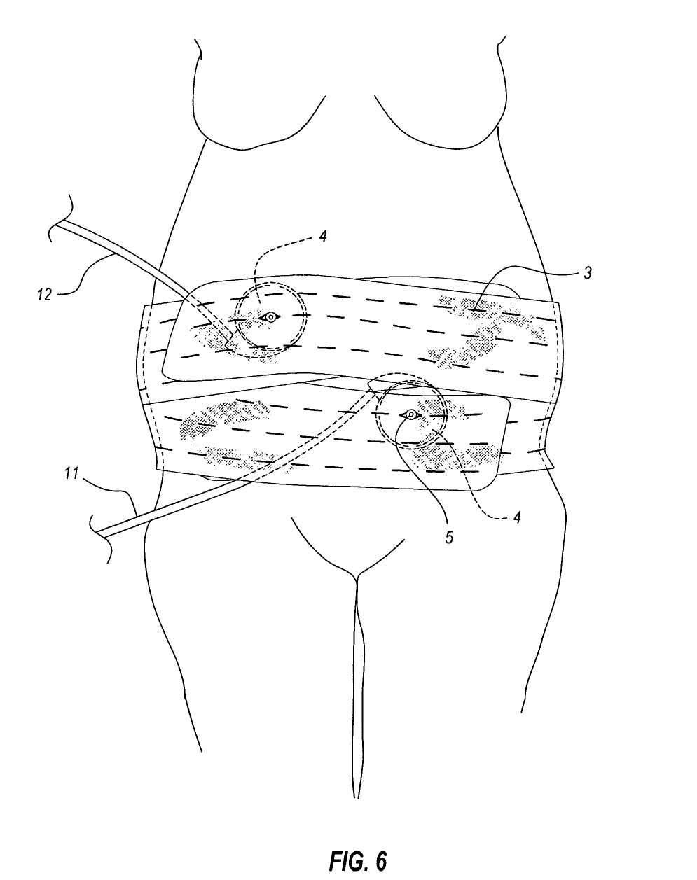

The shape of exemplary transducers 4 are illustrated in FIGS. 5 and 6 and are disc-shaped wherein the face is contacted with the patient's abdomen and the top includes a button-hook 5 for attachment to the belts 1 of the present invention via buttonholes 3.

The transducers 4 of the present invention may be physically connected to the ultrasound machine 7 by a first 11 and second 12 lead as illustrated in FIG. 5 or utilize wireless telemetry to communicate information back to the ultrasound machine 7.

Transducer button-hooks 5 of any size and type may be utilized with the devices of the present invention. Such button-hooks may be fixably or removably attached to transducers 4 or be otherwise connected to a transducer 4 by a medical professional. Some transducers 4 may come with a variety of button-hooks 5 of various sizes and the appropriate size for the buttonholes 3 in the belts 1 of the present invention is easily ascertainable by a medical professional who can then replace the button-hook 5 with the appropriate sized one prior to use.

As illustrated in FIG. 6, the buttonholes 3 allow for placement of the transducers 4 at any location across the patient's abdomen allowing for precise placement and angling of each transducer 4, which in turn, allows for accurate and precise monitoring of the patient's contractions and the fetal heartbeat. One skilled in the art would easily understand how to properly position a transducer 4 in the appropriate buttonhole 3 to provide accurate monitoring of either a mother's contractions or a fetal heartbeat.

The back support 2 of the present invention provides a comfortable way to stabilize the belts 1 and transducers 4 of the present invention during prenatal check-ups, labor and delivery. Any back support 2 may be used with the present invention. The fetal monitoring belts of the present invention may be fixably attached to the back support device or they may be threaded through it in some manner. The back support 2 may support all or a portion of the back of a patient. The back support device 2 may be packaged with the fetal monitoring belts 1 or be a device adapted for this use. One skilled in the art would be familiar with existing devices in the market that may be adapted for use with the fetal monitoring belts 1 of the present invention.

The back support 2 may be made of any material known in the art. In a certain embodiment, the back support is made from a material selected from the group consisting of cotton, canvas, leather, bandaging, foam, solid formed gel, injection-molded plastic or biodegradable materials. In a particular embodiment, the device is made from cotton. The back support 2 may also be cushioned or padded, although versions where no additional cushioning or padding is provided are also contemplated. All such materials are well understood in the medical device industry.

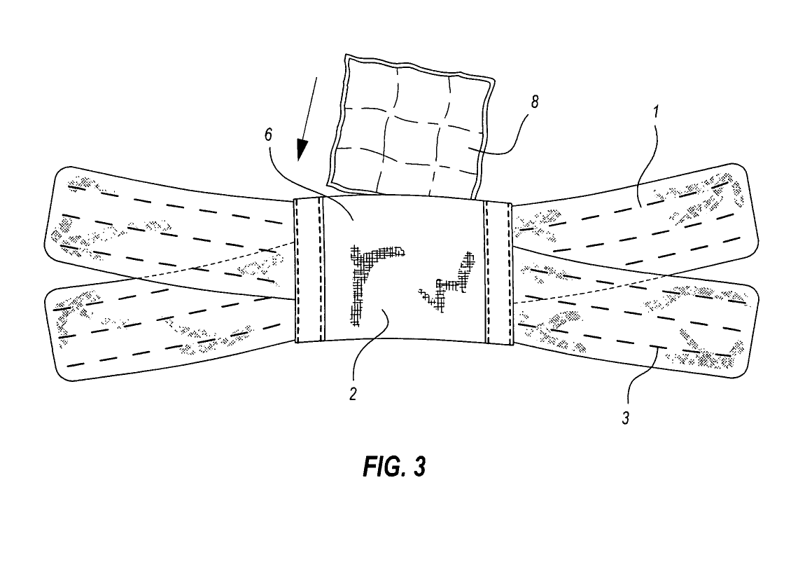

FIG. 3 is a perspective view showing an interior pouch 6 within the back support 2. The interior pouch 6 may be used to hold a therapeutic device 8, such as a hot or cold pack and/or a vibration element, to increase the comfort level of the patient. Such therapeutic device 8 is held snugly in place to the patient's back by the belts 1 circling the patient's torso. In certain embodiments, the interior pouch 6 is open on both the top and bottom to allow for placement of a therapeutic device from both directions. In other embodiments, the interior pouch 6 is sewn on either the top or bottom end to make a pocket-like configuration to hold the therapeutic device 8.

The length and/or width dimensions of the back support 1 may vary depending on the size of the patient, the amount of back support desired and the particular needs of the situation. In certain embodiments, the back support 2 has an elongated and generally rectangular shape adapted to fit about and provide lumbar support for the lower portion of a patient's back, while also providing stabilization for the belts 1 and transducers 4 of the present invention. In general, the length of the back support 2 is defined as the length necessary to span the circumference of the patient's lower back. The width of the back support 2 is generally defined as the width necessary to span the lumbar region of the patient's back. In certain embodiments, the length of the back support 2 can range from ten (10) to forty (40) inches. In a particular embodiment, the length of the back support 2 is sixteen (16) inches. In certain embodiments, the width of the back support 2 can range from about two (2) inches to twelve (12) inches. In a particular embodiment, the back support 2 is seven and a half (7.5) inches wide. In other embodiments, the length and width of the back support 2 is adjustable.

While the illustrative back supports in FIGS. 1-3 has a substantially rectangular shape, it should be understood that the shape may vary from that shown in the figures. In some embodiments, the width of the back support may be greater towards the center portion than at the ends to provide additional support to the center portion of the user's lower back and provide additional stability for the belts 1 of the present invention. One skilled in the art understands how to choose a back support 2 that will provide the most stability for the belts 1 of the present invention that will also be the most comfortable for the patient.

FIG. 5 illustrates an exemplary ultrasound machine 7 for monitoring the rate of a fetal heart beat and also the strength, length and frequency of uterine contractions according to an embodiment of the present invention. As illustrated in FIG. 5, the exemplary ultrasound machine 7 includes a housing 9 provided with processing electronics (not shown), a screen to visualize recordings and a chart recorder 10 to provide a print-out of a patient's uterine contractions or of a fetal heart rate. The ultrasound machine is attached via a first 11 and second 12 lead to transducers 4 attached via button-hooks 5 to the buttonholes 3 on the belts 1 of the present invention. One of the transducers is monitoring the fetal heart rate while the second is monitoring uterine contractions. If there are multiple gestations, multiple monitors must be utilized as each fetal heart rate must be monitored individually.

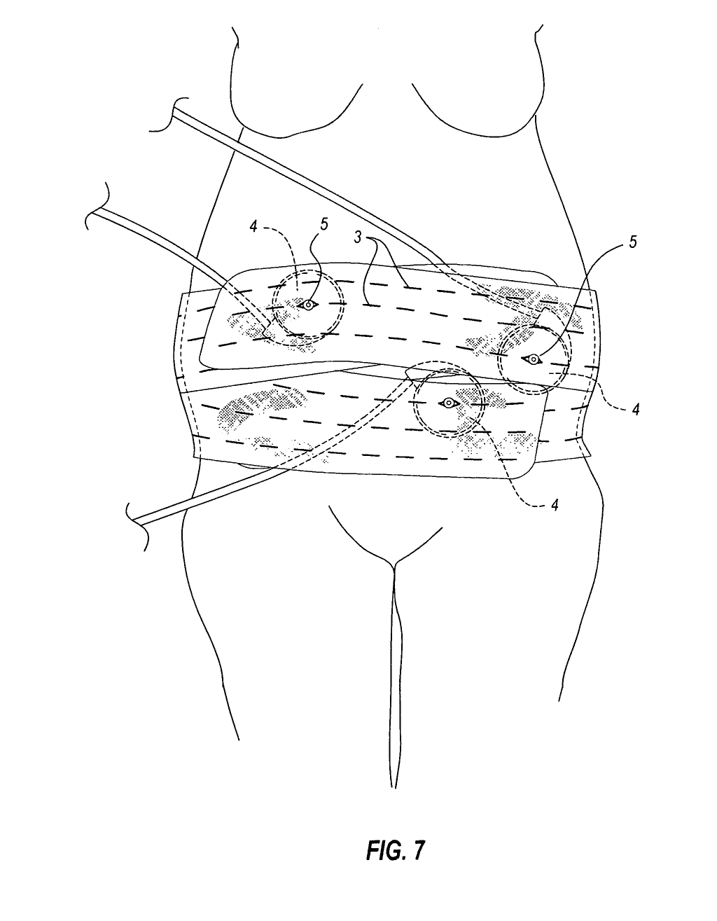

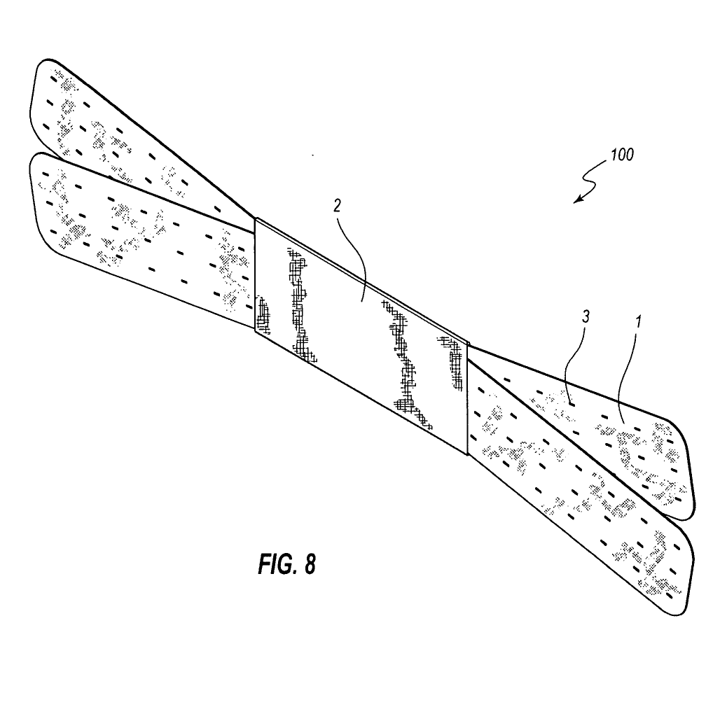

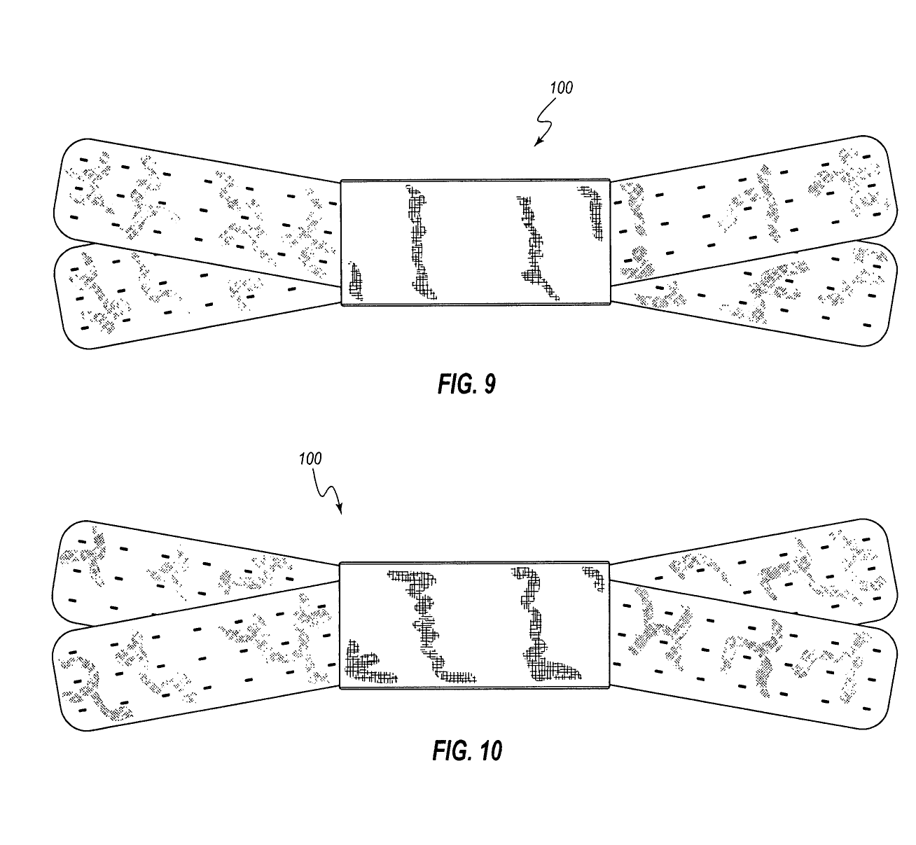

FIG. 7 illustrates an exemplary situation where the patient is carrying twins and two ultrasound machines 7 are needed in order to have two transducers 4 to monitor each of the fetal heart rates. In the situation where there are multiple fetusus, only one transducer 4 is needed to monitor uterine contractions but an individual transducer 4 is needed to monitor each individual fetal heart rate requiring the use of two ultrasound machines 7 as each machine only has two transducers (one for monitoring uterine contractions and one for monitoring one fetal heart rate).

FIGS. 6 and 7 show an exemplary front view of a fetal monitoring belt device illustrating the ease at which transducers 4 can be precisely positioned on a patient due to the multiple rows of buttonholes 3. The buttonholes 3 allow those skilled in the art to easily and precisely position transducers to produce accurate readings of both the fetal heart rate and the patient's uterine contractions.

For the purposes of the present invention, the term “a” or “an” entity refers to one or more than one of that entity; for example, “a belt” or “an transducer” refers to one or more of those elements, or at least one element. As such, the terms “a” or “an”, “one or more” and “at least one” can be used interchangeably herein. It is also to be noted that the terms “comprising”, “including”, and “having” can be used interchangeably. Furthermore, a compound “selected from the group consisting of” refers to one or more of the items in the list that follows, including combinations of two or more of the items.

Citation of the above documents is not intended as an admission that any of the foregoing is prior art. All statements as to the date or representation as to the content of these documents is based on subjective characterization of information available to the applicant, and does not constitute any admission as to the accuracy of the dates or contents of the documents.

All of the METHODS and DEVICES disclosed and claimed herein can be made and executed without undue experimentation in light of the present disclosure. While the compositions and methods of this invention have been described in terms of preferred embodiments, it will be apparent to those of skill in the art that variations may be applied to the METHODS and DEVICES and in the steps or in the sequence of steps of the methods described herein without departing from the concept, spirit and scope of the invention. More specifically, while several different features are described herein with respect to specific embodiments depicted in the drawings, it is contemplated that the fetal belt device of the present invention can include any number of different combinations of features. All such similar substitutes and modifications apparent to those skilled in the art are deemed to be within the spirit, scope and concept of the invention as defined by the appended claims.

We claim:

1. A fetal monitoring belt device comprising:
a) a back support; and
b) one or more belts extending from the back support, wherein said belts maintain the position of at least a first ultrasound transducer and a second ultrasound transducer and wherein said belts carry a tension sufficient to position the face of said first and second transducer in an orientation appropriate to monitor the uterine contractions of a patient or a fetal heartbeat.

2. The fetal monitoring device of claim 1 wherein said belt has a width and a thickness sufficient to maintain its cross-sectional shape while under tension and wrapped around a patient.

3. The fetal monitoring belt device of claim 1 wherein said belts range from three to twenty inches in width.

4. The fetal monitoring belt device of claim 1 wherein said belts range from sixteen to seventy-five inches in length.

5. The fetal monitoring belt device of claim 1 wherein said belts are labeled with an item selected from the group consisting of instructions, advertisements, sizing and color.

6. The fetal monitoring belt device of claim 1 wherein said belts are anchored to or threaded through said back support.

7. The fetal monitoring belt device of claim 1, wherein said belts have buttonholes spaced along the length of the belt.

8. The fetal monitoring belt device of claim 7, wherein said belts have multiple rows of buttonholes.

9. The fetal monitoring belt device of claim 1, wherein said transducers have buttonhooks allowing for direct skin contact with a patient and precise positioning for monitoring uterine contractions or a fetal heartbeat by threading said buttonhooks through said buttonholes in said belts.

10. The fetal monitoring belt device of claim 9, wherein said belts have more than three transducers positioned on them to allow for the monitoring of multiple fetal heart rates.

11. The fetal monitoring belt device of claim 1, wherein said belts are threaded through loops on the sides of transducers and have hook and loop materials on the ends allowing for optimal placement of said transducers.

12. The fetal monitoring belt device of claim 11, wherein said belt further comprises hook and loop materials which can be folded over and attached at the ends.

13. The fetal monitoring belt device of claim 1, wherein said back support further provides an interior pouch.

14. The fetal monitoring belt device of claim 13, wherein said interior pouch includes an item selected from the group consisting of a hot pack, a cold pack and a vibration element.

15. The fetal monitoring belt device of claim 1, wherein said back support is made from a material selected from the group consisting of cotton, canvas, leather, bandaging, foam, solid formed gel, injection-molded plastic or biodegradable materials.
